(19) United States
(12) Patent Application Publication
(10) Pub. No.: US 2020/0121271 A1
(43) Pub. Date: Apr. 23, 2020

(54) Medical Grid with Bar Codes

(71) Applicant: Randall Paul Davis, Eau Claire, WI (US)

(72) Inventor: Randall Paul Davis, Eau Claire, WI (US)

(21) Appl. No.: 15/968,289

(22) Filed: May 1, 2018

(51) Int. Cl. A61B 6/00; A61B 5/055

(52) U.S. Cl. CPC A61B 6/4291; A61B 2090/374; A61B 5/055

(57) ABSTRACT

Medical grid and bar code that can be used on MRI's, CAT Scans, X-rays, and as a reference on any scan. This grid would pinpoint an area or specific location on a person or animal for reference. This grid would be made of vertical and horizontal lines that are either lettered or numbered coordinates. For example, a swimmers cap could have a grid with the X,Y,Z coordinates printed in order to pinpoint the accuracy of the area requiring attention. The barcodes could be used for personal identification.

### BACKGROUND OF THE INVENTION

Medical Grid with Bar Codes:

I have personally had over 200 MRI's and several of them have been overlapped due to stopping and starting the MRI and placing the disc above or below the actual herniated disc. There is a need for accurate MRI's, CAT Scans, X-rays, etc. for the benefit of the patient due to the collaboration of medical facilities. This would be a tool that could also be utilized during medical lawsuit situations. This invention would pinpoint the area of the body that needs medical attention.

### BRIEF SUMMARY OF THE INVENTION

Medical grid and bar code that can be used on MRI's, CAT Scans, X-rays, and as a reference on any scan. This grid would pinpoint an area or specific location on a person or animal for reference. This grid would be made of vertical and horizontal lines that are either lettered or numbered coordinates. For example, a swimmers cap could have a grid with the coordinates printed in order to pinpoint the accuracy of the area requiring attention.

### REPLACEMENT BRIEF DESCRIPTION OF THE DRAWINGS

FIG. 1 is a top view of my invention showing an example of a grid and barcodes on a person's back.

FIG. 2 is a side view of grid and barcodes thereof:

FIG. 3 is a top view of the grid thereof: the bottom being a mirror image thereof:

FIG. 4 is a side elevation view thereof: the opposite side being a mirror image thereof:

FIG. 5 is an end view thereof: the opposite end being a mirror image thereof:

FIG. 6 is a top view of the barcode thereof: the bottom being a mirror image of thereof:

FIG. 7 is a side elevational view thereof:

FIG. 8 is an end view thereof:

FIG. 9 is a side view of a swimmers cap grid on a person's head thereof: the opposite side being a mirror image thereof:

FIG. 10 is a side elevational view of a swimmers cap grid thereof: the opposite side being a mirror image thereof:

FIG. 11 is a top view thereof: the bottom being a mirror image thereof:

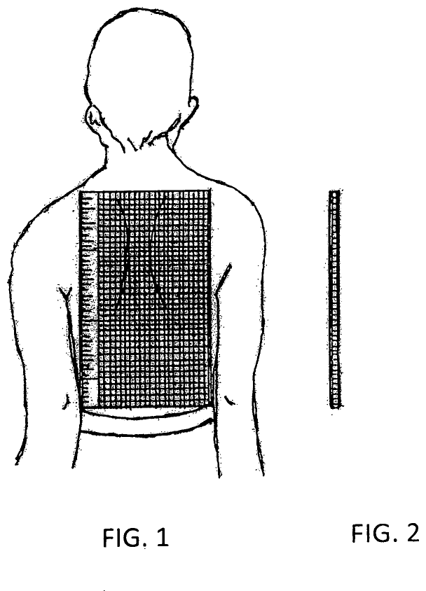

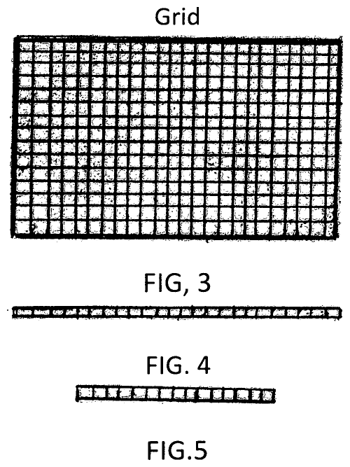

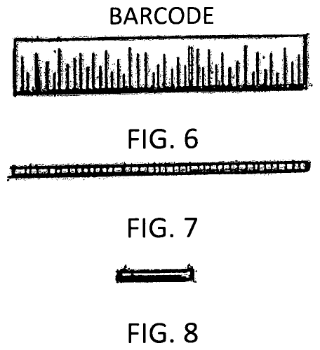

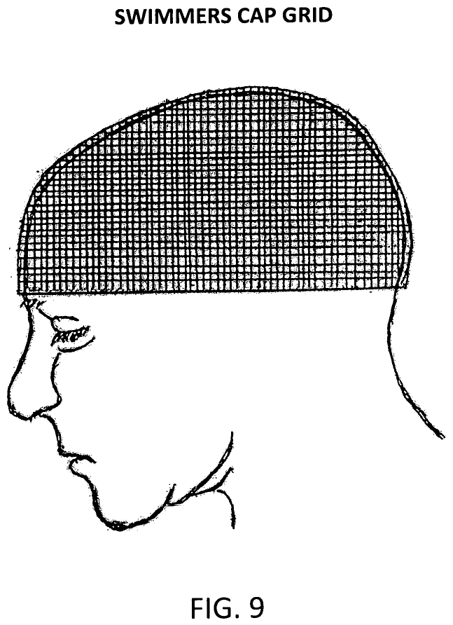

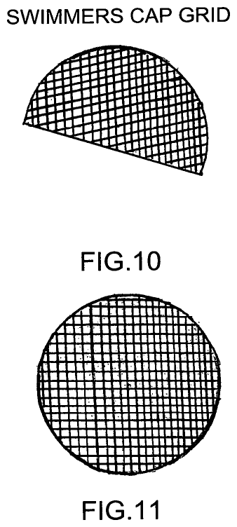

### DETAILED DESCRIPTION OF THE INVENTION

Medical grids could be used on any scan or X-ray as a reference point to ensure accurate scans. The grids could come in a variety of shapes and sizes to meet the needs of the individual or animal. The grids could be used on any body part: head, back, arms, legs, etc. The adhesive used to keep the grid in place could simply be medical tape. The grid itself could also have adhesive on the back that allows it to stick to the skin or fabric and then peeled off clean. The grid could be made of plastic, film, cloth, paper, or any non-metallic material. The grid identifiers would be lines, numbers, or letters to make up coordinates. A barcode on the grid would be used for identification of the scan or individual scanned. This barcode is also used to prevent overlapping by scanning the barcode after the procedure. The ink or adhesive on the grid would be made of a mixture of powdered bone from animals or calcium phosphate and/or carbonate along with ink. This would allow the grid to appear on the scans during the procedure. A chiropractor could use a clear grid with regular ink which can be used as a reference utilizing vertical and horizontal guides. Again, this medical grid could be used on animals as well. This grid could potentially be mandated on all workman's compensation claims.

1-5. (canceled)

6. A grid, barcode and swimmers cap grid. Printed or adhered to film or any material.

7. The lines of the grid and barcode of claim 6 wherein is comprised of Calcium Carbonate phosphate or powdered bone with adhesive.

8. A grid, barcode and swimmers cap listed in claim 6, is lettered, numbered, or X,Y,Z coordinates.

9. A grid, barcode and swimmers cap listed in claim 6, will include different width, lengths and thicknesses.
